(19) United States
(12) Patent Application Publication
(10) Pub. No.: US 2018/0110804 A1
(43) Pub. Date: Apr. 26, 2018

(54) Burn, scar, and wound treatment creams

(71) Applicant: NuGene, Inc., Irvine, CA (US)

(72) Inventors: Sanjay Dhar, Irvine, CA (US); Mohammad Ali Kharazmi, Corona Del Mar, CA (US); Mohammad Saeed Kharazmi, Irvine, CA (US)

(21) Appl. No.: 15/555,968

(22) PCT Filed: Feb. 3, 2016
(86) PCT No.: PCT/US16/16463

§ 371 Date: Sep. 5, 2017

Related U.S. Application Data
(60) Provisional application No. 62/112,124, filed on Feb. 4, 2015.

(51) Int. Cl. A61K 33/38; A61K 9/06; A61K 35/35; A61K 47/44; A61K 31/355; A61K 36/889

(52) U.S. Cl. CPC A61K 33/38; A61K 9/06; A61K 36/889; A61K 47/44; A61K 31/355; A61K 35/35

(57) ABSTRACT

Dermal treatment compositions include an emollient base, a conditioned medium, and a nanosilver particulate. The conditioned medium may be harvested from culture of human adipocyte-derived stem cells. The stem cells may be cultured in the presence of the nanosilver particulate. In other embodiments, the stem cells are cultured in medium that does not contain a nanosilver particulate. Instead the conditioned medium is compounded with the nanosilver particulate after the media is harvested from the stem cell culture.

### CROSS-REFERENCE TO RELATED APPLICATIONS

This application claims priority to U.S. Provisional Application 62/112,124 filed 4 Feb. 2015, the disclosure of which is incorporated by reference.

### BACKGROUND OF THE INVENTION

### Field of the Invention

This invention is in the field of dermal care and treatment. In particular, it concerns compositions that improve the condition of wounds, scars, or burns by topical application.

### Human Adipose-Derived Stem Cell Culture Medium

Many tissues, including living layers of the skin, respond to appropriate mixtures of growth factors to encourage regeneration. For example US 2012/0065129 A1 to Park asserts that a culture medium of adipose-derived stem cells and growth factors isolated from the culture medium can be advantageously applied in drugs, quasi drugs, and cosmetics for wound healing. PCT US2014/034738, commonly assigned with this application, describes dermal treatment compositions including a medium recovered from human adipose-derived stem cell culture (HADSCC).

### Nanosilver

Silver is an effective killing agent against many types of bacteria, viruses, and fungi. It is widely used as a microbicide, as a preservative, and as a deodorant. Both preservative and deodorant properties are commonly ascribed to its microbicidal activity. Silver may also enhance the antibacterial activity of various antibiotics such as penicillin, erythromycin, and vancomycin. Reduced silver particles with size range below about 100 nm are commonly called nanosilver. Nanosilver particles frequently are capped with surface chemical groups that support their stability and dispersion in aqueous suspension.

Nanosilver particles act as antimicrobial and antifungal agents in applications including sanitizing sprays, textiles, solid surfaces, and cleaning devices. Some of these applications include close human contact (e.g. wound dressings, contact lenses, socks, pillows, cellular phones, and shampoos) and even ingestion (toothpastes). Nanosilver may be deposited or coated onto medical devices or fabrics to make them suitable for controlling infections.

Nanosilver dispersions have anti-inflammatory activity in human application; nanosilver may alter the expression of enzymes that are important in inflammatory and tissue repair processes, such as matrix metallo-proteinases. In addition, nanosilver can modulate cytokines involved in wound healing such as by suppressing expression of interleukin (IL)-12, and IL-1 and of TNF-α, and it may induce apoptosis of inflammatory cells. The magnitude of the anti-inflammatory effects of nanosilver is related to the exposure of the treated tissue to reduced silver atoms and thus on a per gram basis depends on the size of the nanosilver particulates. The enhanced effects from smaller particles may be greater than linear with surface area as particles approach a few hundred atoms due to quantum confinement effects. To some extent the effects also depend on the shape of the particles as different shapes expose different crystalline planes with different packing density of silver atoms. The size producing an optimum combination of properties depends on the particular microenvironment, the tissue treated, and the effect measured.

Wound dressings containing nanosilver cause wounds to heal more quickly. Nanosilver is used extensively in wound management, particularly in burn treatment. Other reported applications include chronic wounds, burns injuries in children and neonates, ulcers (including diabetic ulcers, rheumatoid arthritis-associated leg ulcers, and venous ulcers), toxic epidermal necrolysis, healing of donor sites, and meshed skin grafts.

Nanosilver particulates, like any material having a chemical effect on the body, also have negative effects. These include reported alveolar macrophage toxicity that increases as particle size decreases. In studies of cultured cells, nanosilver particulates “significantly inhibited” keratinocyte proliferation, and affected cell morphology.

### BRIEF SUMMARY OF THE INVENTION

In some embodiments, the invention includes a dermal treatment composition that has an emollient base, a conditioned medium; and a nanosilver particulate. The conditioned medium may be dispersed in the emollient base; the nanosilver particulate may be suspended in the conditioned medium.

The conditioned medium may harvested from culture of human adipocyte-derived stem cells. The stem cells may be cultured in the presence of the nanosilver particulate. The culture medium may include one of Dulbecco's Modified Eagle's Medium or RPMI 1640 medium containing 0.0025-0.5% nanosilver particulate by weight of the medium.

In other embodiments, the stem cells are cultured in medium that does not contain a nanosilver particulate. Instead the conditioned medium is compounded with the nanosilver particulate after the media is harvested from the stem cell culture.

In any of these embodiments, the conditioned medium may form about 40% by weight of the composition. The term about 40% should be understood to include variations that depend on the details of the cell culture. The 40% amount refers to medium harvested approximately according the methods described in PCT US2014/034738 referenced above (a time between medium changes less than about once every 72 hours and preferably about once every 48 hours with cell growth at about 40% to about 90% confluence). The sections of PCT US2014/034738 describing conditioned medium are hereby incorporated by reference. Medium exposed to cells for a shorter period of time, or to a lower concentration of growth phase cells, may be present in higher weight in the composition. Medium that has been exposed to cells for a longer time or that has been concentrated, may be present at a lower weight in the composition.

In any of these embodiments, the nanosilver particulate may form about 0.001-0.2% by weight of the composition. The nanosilver particulate includes a stabilizing coating, such as a coating including one or more of citrate, tannic acid, polyvinylpyrrolidone, silica, polyethylene glycol, oligonucleotides, or a peptide.

In any of these embodiments, the nanosilver particulate may include silver particles have a size range of about 1-10 nm. The nanosilver particulate may form about 0.005% by weight of the composition.

The emollient base may include an oil and an emulsifying agent. The oil may form about 5-20% by weight of the composition and the emulsifying agent may form about 10-20% of the composition. Oil may include one or more of coconut oil, avocado oil, neem oil, rosemary oil, manuka oil, safflower oil, or geranium oil. In some embodiments, the composition may include at most one of vitamin E or coconut oil. Either coconut oil or Vitamin E, if present, may form about 1-5% of the weight of the composition. The balance of the composition may include other materials such as those detailed in the Examples.

In other embodiments, the invention includes a burn treatment composition consisting essentially of about 5-20% by weight of an oil, about 10-20% by weight of an emulsifying agent, and about 40% by weight of a conditioned medium harvested from culture of human adipocyte-derived stem cells, where the stem cells are cultured in the presence of a nanosilver particulate such that the nanosilver particulate forms about 0.001-0.2% by weight of the composition. The oil, the emulsifying agent, and the conditioned media may form an emulsion.

The burn treatment composition may further include about 1-5% vitamin E. Any of these compositions may also be applicable to lessen the appearance of scars.

In still other embodiments, the invention includes a wound treatment composition consisting essentially of about 5-20% by weight of an oil, about 10-20% by weight of an emulsifying agent, and about 40% by weight of a conditioned medium harvested from culture of human adipocyte-derived stem cells, where the stem cells are cultured in the presence of a nanosilver particulate such that the nanosilver particulate forms about 0.001-0.2% by weight of the composition.

The wound treatment may further include about 1-5% coconut oil.

### DETAILED DESCRIPTION OF THE INVENTION

Nanosilver particles may be produced in a variety of sizes known in the art. In some embodiments, nanosilver particles may be in the size range of about 1-10 nm. Nanosilver particles may be capped with any of a variety of stabilizing coatings including citrate, tannic acid, polyvinylpyrrolidone, silica, polyethylene glycol, oligonucleotides, or peptides.

Adipose-derived stem cells are stem cells extracted from adipose tissues. Adipose tissue, like other tissue types, is not a homogenous mixture of a single cell type. Instead adipose tissue includes a combination of fat cells, vasculature, connective tissue, and blood cells. Human adipose tissue is available ex vivo as a result of cosmetic procedures including liposuction. Stem cells may be extracted from such tissue by any of a number of methods known in the art, including treatment with surfactants or enzymes (including proteases such as such as collagenase, or trypsin), maceration, separation by centrifugation, filtering, or settling, ultrasonic treatment, adherent culturing, or some combination of these methods.

Growth of such adipose-derived stem cells includes supply of nutrients for the cells through provision of an aqueous culture medium. Cells grow in culture in contact with medium and extract nutrients from it. These cells also deliver to the medium products of their growth and metabolism. Among the products are growth factors and cytokines as well as metabolic products. Conventional tissue culture requires replacement of culture medium as cells use up the nutrients and deliver products that may affect future cell growth. This replacement may be either continuous, with a portion of the medium removed as new medium is added, or intermittent with periodic replacement of some or all of the culture medium in a vessel. Culture medium removed after exposure to cells in culture is known as spent or conditioned medium.

Culture medium compositions typically include essential amino acids, salts, vitamins, minerals, trace metals, sugars, lipids, and nucleosides. Cell culture medium attempts to supply the components necessary to meet the nutritional needs required to grow cells in a controlled, artificial and in vitro environment. Nutrient formulations, pH, and osmolarity may vary depending on the type of cell cultured, on cell density, and on the culture system employed. The scientific literature includes description of many cell culture medium formulations; a number of such media are commercially available. Conditioned medium contains many of the original components of the medium, as well as a variety of cellular metabolites and secreted proteins, including, for example, biologically active growth factors, inflammatory mediators, other extracellular proteins, peptides, and other materials. Examples of suitable culture media are Dulbecco's Modified Eagle's Medium and RPMI 1640. Such media may be supplemented by other nutrients, growth supporting materials, or antibiotics as is known in the art. An exemplary process of producing a human adipose derived stem cell conditioned (“HADSCC”) medium is that described in PCT US2014/034738, commonly assigned with this application.

The presence of cytokines, growth factors, and other materials produced by the cultured adipose-derived stem cells may affect the continued growth of the cells and the subsequent production of growth factors by the cells. Thus the mix and concentration of the cellular products has a complex dependence on the stage of growth of the cultured cells and the length of time between medium changes. Some early produced products may be subsequently consumed or destroyed during later stages of culture. Others may be increased in concentration, and new materials may be produced as secondary effects of the products on the growing cells. In some embodiments, as described in PCT US2014/034738, the culture medium may be harvested from adherent cultures having from about 40% to about 90% confluence. Culture confluence of about 70% may be especially suitable for use in wound and burn treatment preparations.

In some embodiments the burn creams and wound creams of the invention include a HADSCC medium where the media is supplemented with nanosilver particulates before exposure to the human adipose-derived stem cell culture. In these embodiments, the stem cells are cultured in the presence of the nanosilver particulates. Just as nanosilver can affect the healing of damaged tissue when applied during wound healing or burn healing, so also may nanosilver affect the growth of human adipose-derived stem cells in culture and their production of stem cell products including growth factors, cytokines, stress proteins, and nutrients. Thus HADSCC medium where the stem cells are cultured in the presence of the nanosilver particulates (“HADSCC silver medium”) may contain a different mix of stem cell products than HADSCC medium where the stem cells are cultured in the absence of the nanosilver particulates (“HADSCC non-silver medium”). This mix may be particularly appropriate for dermal healing applications because it includes a mix of cell products from cells in a growth stage that approximates that of early healing skin. In some embodiments where the media is supplemented with nanosilver particulates before exposure to the human adipose-derived stem cell culture cells, the media may be otherwise free of or have reduced amounts of antimicrobials such as antibiotics. HADSCC silver medium (other than the presence of nanosilver and possible absence of antibiotics) may be prepared by a similar process to that of HADSCC non-silver media.

In other embodiments the burn creams and wound creams of the invention include a HADSCC medium where the media is compounded with nanosilver particulates after the media is harvested.

Both embodiments containing HADSCC silver media and those containing HADSCC non-silver medium media compounded with nanosilver particulates after the media is harvested (“HADSCC post-harvest silver medium”) contain nanosilver particulates as well as stem cell products, but the mixture of stem cell products may differ because of the effect of the nanosilver particulates during the growth of the stem cells.

In addition to HADSCC post-harvest silver media (or HADSCC silver media), embodiments of the invention include support ingredients such as oils, vitamins, and emulsifying agents. Oils may include any of a variety of oils helpful to form an emulsion with the aqueous media components. An emulsion is advantageous because it delivers both oil soluble and aqueous soluble constituents to a dermal site and because it can reduce run off and evaporation. Exemplary oils include coconut oil, avocado oil, neem oil, rosemary oil, manuka oil, safflower oil, and geranium oil. Emulsifying agents may include commercial cold process waxes such as Jeesperse® CPW-CG-T (a mixture of cetyl alcohol, sodium acrylate, sodium acryloyl dimethyl taurate copolymer, glyceryl monostearate, and caprylic triglyceride). Jeesperse is a registered trademark of Jeen International Corporation of Fairfield, N.J. Other support ingredients may also be present.

In some embodiments the burn creams differ from the wound creams by the inclusion of vitamin E (Tocopheryl Acetate) in the burn cream but not in the wound cream or by the inclusion of coconut oil in the wound cream but not in the burn cream. Vitamin E is the principle fat-soluble antioxidant in skin and is depleted in burns; oxidative injury contributes to burn damage. Admixture of vitamin E to a burn cream may serve to at least partially supplement the depleted antioxidants in burned areas of skin. Coconut oil, which may be in the form of virgin coconut oil, reportedly provides more rapid reepithelialization and neovascularization, with higher turnover of collagen. See Nevin et al. Skin Pharmacology and Physiology 23(6) pp. 290-297, 2010.

We have found that a suitable mixture contains about 40% by weight of HADSCC post-harvest silver media or HADSCC silver media. Nanosilver may be present to a final concentration of about 0.001-0.2%. Oils may range from 5 to about 20% with emulsifying agents in the range of 10-20% of the mixture. The balance may be other aqueous ingredients such as aloe vera gel or other plant extracts or sterile water.

The wound creams and burn creams of the invention may be applied in a manner typical of conventional wound and burn care products. For example, the wound creams and burn creams may be directly applied to the surface of damaged skin or may be first applied to a dressing with the dressing applied to the skin.

Without intent to be bound by theory, Applicant believes that the beneficial effects of combining nanosilver particles and HADSSC media may arise because the mix of stem cell products from HADSSC media or from HADSSC silver media complements the nanosilver-activated wound healing response. HADSSC silver media may include especially complementary stem cell products. Further, nanosilver particulates have in some instances been associated with cytotoxic effects. These negative effects of nanosilver may be at least in part ameliorated by the growth factors contained in the HADSSC medium.

### Example 1 Composition of Wound Cream or Scar Cream

The listed ingredients (with weighed quantities adjusted for batch size) are typically combined in a cleaned and sterilized tank with moderate mixing or in smaller batches in sterile disposable labware. The ingredients may be added in the order listed, one at a time, mixing well between additions. The ingredients may be subject to mechanical emulsification during processing. In some embodiments, the first two listed ingredients may be combined as HADSCC silver media.

| | % by weight |
|---|---|
| | of final |
| Ingredient | mixture |
| HADSCC Media | 40% |
| Nanosilver particulates (1-10 nm, citrate capped) | 0.001-0.2% |
| Jeesperse ® CPW-CG-T (Cetyl Alcohol/Sodium | 10-15% |
| Acrylate/Sodium Acryloyl Dimethyl Taurate | |
| Copolymer/Glyceryl Monostearate/Caprylic/Triglyceride) | |
| Butylene Glycol | 5-10% |
| Niacinamide (Vitamin B3) | 5-10% |
| DL-Pantothenic (Vitamin B-5) | 1-5% |
| Tetrahexyldecyl Ascorbate (Vitamin C Oily) | 1-4% |
| Glycolic Acid 70% | 5-10% |
| Zinc Oxide/Triethoxycaprylylsilane/C12-15 Alkyl | 2-5% |
| Benzoate/DEA Oleth 3 Phosphate | |
| Borage Water (1%) | 1-3% |
| Salicylic Acid | 1-3% |
| Willow Bark Extract | 1-3% |
| Witch Hazel | 1-3% |
| Lavender Extract | 1-3% |
| Aloe Vera Gel | 1-5% |
| Avocado Oil | 0.1-0.5% |
| Coconut Oil | 1-5% |
| Echinacea Extract | 1-5% |
| Golden Seed Extract | 1-3% |
| Manuka Oil | 0.1-1.5% |
| Phenoxyethanol/Sorbic Acid/Caprylyl Glycol | 1-3% |
| Tetrapeptide-14 | 1-3% |
| Grapefruit Seed Extract | 1-3% |
| Green Tea Extract | 0.1-1.5% |
| Cellulose Gum | 0.1-1.5% |
| Fragrance | 0.1-1.5% |
| Calendula Extract | 0.1-1.5% |
| Allantoin | 0.01-0.1% |
| Nanosome Copper Peptides | 0.001-0.01% |
| EGF-1 (Human Oligopeptide-1) | 0.001-0.01% |
| Safflower Oil | 1-5% |
| Geranium Oil | 0.1-0.5% |
| Slippery Elm | 1-3% |

### Example 2 Composition of Burn Cream

The listed ingredients (with weighed quantities adjusted for batch size) are typically combined in a cleaned and sterilized tank with moderate mixing or in smaller batches in sterile disposable labware. The ingredients may be added in the order listed, one at a time, mixing well between additions. The ingredients may be subject to mechanical emulsification during processing. In some embodiments, the first two listed ingredients may be combined as HADSCC silver media.

| | % by weight |
|---|---|
| | of final |
| Ingredient | mixture |
| HADSCC Media | 40% |
| Nanosilver particulates (1-10 nm, citrate capped) | 0.001-0.2% |
| Jeesperse ® CPW-CG-T (Cetyl Alcohol/Sodium | 10-15% |
| Acrylate/Sodium Acryloyl Dimethyl Taurate | |
| Copolymer/Glyceryl Monostearate/Caprylic/Triglyceride) | |
| Butylene Glycol | 5-10% |
| Niacinamide (Vitamin B3) | 5-10% |
| DL-Pantothenic (Vitamin B-5) | 1-5% |
| Tocopheryl Acetate (Vitamin E) | 1-5% |
| Tetrahexyldecyl Ascorbate (Vitamin C Oily) | 1-4% |
| Glycolic Acid 70% | 5-10% |
| Zinc Oxide/Triethoxycaprylylsilane/C12-15 Alkyl | 2-5% |
| Benzoate/DEA Oleth 3 Phosphate | |
| Borage Water (1%) | 1-3% |
| Salicylic Acid | 1-3% |
| Willow Bark Extract | 1-3% |
| Witch Hazel | 1-3% |
| Lavender Extract | 1-3% |
| Aloe Vera Gel | 1-5% |
| Avocado Oil | 0.1-0.5% |
| Echinacea Extract | 1-5% |
| Golden Seed Extract | 1-3% |
| Manuka Oil | 0.1-1.5% |
| Phenoxyethanol/Sorbic Acid/Caprylyl Glycol | 1-3% |
| Tetrapeptide-14 | 1-3% |
| Grapefruit Seed Extract | 1-3% |
| Green Tea Extract | 0.1-1.5% |
| Cellulose Gum | 0.1-1.5% |
| Fragrance | 0.1-1.5% |
| Calendula Extract | 0.1-1.5% |
| Allantoin | 0.01-0.1% |
| Nanosome Copper Peptides | 0.001-0.01% |
| EGF-1 (Human Oligopeptide-1) | 0.001-0.01% |
| Safflower Oil | 1-5% |
| Geranium Oil | 0.1-0.5% |
| Slippery Elm | 1-3% |

This specification discloses various aspects of the invention with reference to particular embodiments, but it should be understood that any of the features, functions, materials, or characteristics may be combined with any other of the described features, functions, materials, or characteristics. The description of particular features, functions, materials, or characteristics in connection with a particular embodiment is exemplary only; it should be understood that it is within the knowledge of one skilled in the art to include such feature, structure, or characteristic in connection with other embodiments whether or not explicitly described. We intend the scope of the appended claims to encompass such alternative embodiments. Variations on these described embodiments will become apparent to those of ordinary skill in the art upon reading the description. The inventors expect skilled artisans to employ such variations as appropriate, and the inventors intend for the invention to be practiced otherwise than specifically described herein. Accordingly, this specification and claims include all modifications and equivalents of the subject matter recited in the claims appended hereto as permitted by applicable law.

Unless otherwise indicated, all numbers used in the specification and claims are to be understood as being modified in all instances by the term “about.” Unless indicated to the contrary, the numerical values in the specification and attached claims are approximations that may vary depending upon the desired properties sought to be obtained.

The terms “a,” “an,” “the” and similar referents used in the context of describing the invention (especially in the context of the following claims) are intended to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the claims. No language in the specification should be construed as indicating any non-claimed element essential to the practice of the invention.

1. A dermal treatment composition comprising:
an emollient base;
a conditioned medium dispersed in the emollient base; and
a nanosilver particulate suspended in the conditioned medium.

2. The composition of claim 1, wherein the conditioned medium is harvested from culture of human adipocyte-derived stem cells.

3. The composition of claim 2, wherein the stem cells are cultured in the presence of the nanosilver particulate.

4. The composition of claim 3, wherein the stem cells are cultured in one of Dulbecco's Modified Eagle's Medium or RPM11640 medium containing 0.0025-0.5% nanosilver particulate by weight of the medium.

5. The composition of claim 2, wherein the conditioned medium is compounded with the nanosilver particulate after the media is harvested.

6. The composition of claim 1, wherein the conditioned medium forms about 40% by weight of the composition.

7. The composition of claim 1, wherein the nanosilver particulate forms about 0.001-0.2% by weight of the composition.

8. The composition of claim 4, wherein the nanosilver particulate includes a stabilizing coating.

9. The composition of claim 5 wherein the stabilizing coating including one or more of citrate, tannic acid, polyvinylpyrrolidone, silica, polyethylene glycol, oligonucleotides, and a peptide.

10. The composition of claim 4, wherein the nanosilver particulate includes silver particles have a size range of about 1-10 nm, and wherein the nanosilver particulate forms about 0.005% by weight of the composition.

11. The composition of claim 1, wherein the emollient base includes an oil and an emulsifying agent.

12. The composition of claim 11, wherein the oil forms about 5-20% by weight of the composition and the emulsifying agent forms about 10-20% of the composition.

13. The composition of claim 12, wherein the oil includes one or more of coconut oil, avocado oil, neem oil, rosemary oil, manuka oil, safflower oil, or geranium oil.

14. The composition of claim 1 further comprising at most one of vitamin E and coconut oil.

15. A burn treatment composition consisting essentially of:
about 5-20% by weight of an oil;
about 10-20% by weight of an emulsifying agent;
about 40% by weight of a conditioned medium harvested from culture of human adipocyte-derived stem cells, the stem cells cultured in the presence of a nanosilver particulate such that the nanosilver particulate forms about 0.001-0.2% by weight of the composition,
wherein the oil, the emulsifying agent, and the conditioned media form an emulsion.

16. The burn treatment composition of claim 15 further comprising about 1-5% vitamin E mixed with the oil.

17. A wound treatment composition consisting essentially of:
about 5-20% by weight of an oil;
about 10-20% by weight of an emulsifying agent;
about 40% by weight of a conditioned medium harvested from culture of human adipocyte-derived stem cells, the stem cells cultured in the presence of a nanosilver particulate such that the nanosilver particulate forms about 0.001-0.2% by weight of the composition.

18. The wound treatment composition of claim 15, wherein the oil includes about 1-5% coconut oil.
